(19) United States
(12) Plant Patent Application Publication
(10) Pub. No.: US 2015/0351302 P1
(43) Pub. Date: Dec. 3, 2015

(54) Dianthus plant named 'KLEDG13159'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/120,504

(22) Filed: May 27, 2014

(51) Int. Cl. A01H 5/00

(52) U.S. Cl. PLT/283

(57) ABSTRACT

A new Dianthus plant particularly distinguished by single-type flowers, raspberry-red flower color, and an upright and bushy plant habit, is disclosed.

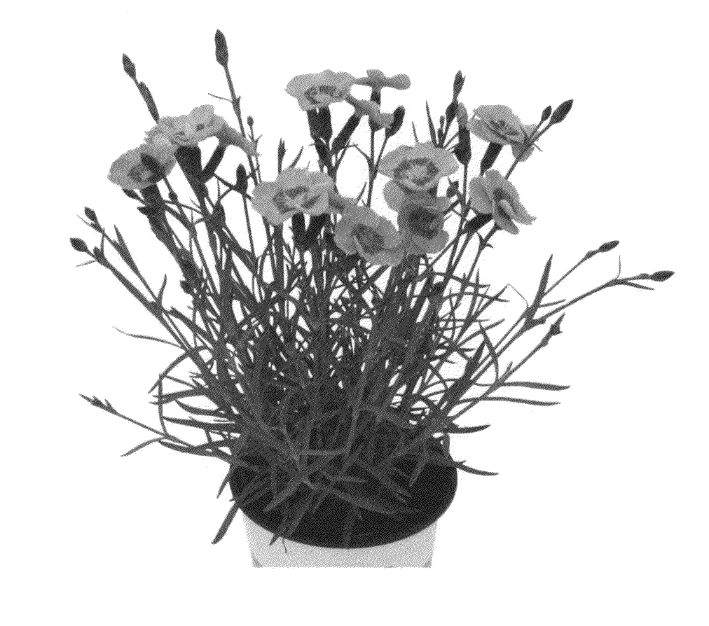

### GENUS AND SPECIES

Dianthus sp.

### VARIETY DENOMINATION

‘KLEDG13159’

### BACKGROUND OF THE NEW PLANT

The present invention comprises a new and distinct variety of Dianthus, botanically known as Dianthus sp., and hereinafter referred to by the variety name ‘KLEDG13159’. ‘KLEDG13159’ originated from a cross-pollination between the proprietary female Dianthus parent ‘DSS 2007 0031’ (unpatented) and proprietary male Dianthus parent ‘DSS 2007 0026’ (unpatented) in June 2009 in Stuttgart, Germany.

The seeds from the cross were sown and plants were grown in a greenhouse for evaluation, where an individual plant designated ‘KLEDG13159’ was selected from the group of plants June 2010 in Stuttgart, Germany. In June 2010, ‘KLEDG13159’ was first vegetatively propagated by vegetative cuttings. ‘KLEDG13159’ was found to reproduce true to type in successive generations of asexual propagation in Stuttgart, Germany via vegetative cuttings.

### SUMMARY

The following are the most outstanding and distinguishing characteristics of this new variety when grown under normal horticultural practices in Stuttgart, Germany.

1. Single-type flowers having a raspberry-red flower color; and

2. An upright and bushy plant habit.

### CROSS-REFERENCE TO RELATED APPLICATIONS

A Plant Breeders Rights application was filed in Canada on Jan. 20, 2014, Application Number 14-8173. ‘KLEDG13159’ has not been sold or made publicly available more than one year prior to the filing date of the present application.

### DESCRIPTION OF THE PHOTOGRAPH

This new Dianthus plant is illustrated by the accompanying photograph of a plant approximately 4 to 5-months old and grown in a glass greenhouse in Stuttgart, Germany. The photograph shows the whole plant, including the flowers and foliage and buds and was taken in June 2012 in Stuttgart, Germany. The colors shown are as true as can be reasonably obtained by conventional photographic procedures.

### DESCRIPTION OF THE NEW VARIETY

The following detailed descriptions set forth the distinctive characteristics of ‘KLEDG13159’. The data which define these characteristics were collected from asexual reproductions carried out in Stuttgart, Germany. Data was collected on 4-month old plants grown in a greenhouse in March 2014. Color references are to The R.H.S. Colour Chart of The Royal Horticultural Society of London (R.H.S.), 5^{th} edition (2007).
Classification:
   Family.—Caryophyllaceae.
   Botanical.—Dianthus sp.
   Common.—Garden carnation.
   Designation.—‘KLEDG13159’.
Parentage:
   Female parent.—The proprietary Dianthus variety ‘DSS 2007 0031’ (unpatented).
   Male parent.—The proprietary Dianthus variety ‘DSS 2007 0026’ (unpatented).
Plant:
   Propagation type.—Vegetative cuttings.
   Time to produce a rooted young plant.—20 days.
   Root description.—White, well-branched, and fine.
   Rooting habit.—Lanceolate.
   Plant form.—Potted and garden plant, upright and bushy.
   Plant height.—17.0 cm.
   Plant diameter or spread.—17.0 cm.
Lateral branches:
   Length.—7.0 cm.
   Diameter.—2.0 cm.
   Internode length.—1.5 cm.
   Node length.—0.5 cm.
   Node diameter.—0.4 cm.
   Texture.—Smooth.
   Color.—RHS 139C.
Foliage:
   Arrangement.—Opposite.
   Length.—6.0 cm.
   Width.—0.4 cm.
   Shape.—Lanceolate.
   Apex.—Acute.
   Base.—Obtuse.
   Margin.—Entire.
   Texture (both upper and lower surfaces).—Smooth.
   Immature leaf color.—Upper surface: RHS 139C Lower surface: RHS 139D.
   Mature leaf color.—Upper surface: RHS 139C Lower surface: RHS 139D.
   Venation pattern.—Pinnate.
   Venation color (both upper and lower surfaces).—RHS 139C.
Flowers:
   Flower type and habit.—Most flowers are single, but some flowers are also semi-double.
   Fragrance.—Present.
   Natural flowering season.—Spring to fall.
   Flower longevity.—7 to 9 days.
   Buds.—Length: 1.5 cm Diameter: 0.5 cm Shape: Tall cylinder to ovate Color: RHS 187A.
   Corolla.—Quantity of petals and arrangement: 5 arranged radially Corolla height: 1.3 cm Petal length: 3.5 cm Petal width: 2.0 cm Petal shape: V-shaped Petal apex: Obtuse Petal base: Cuspidate Petal margin: Serrate Petal texture (both upper and lower surfaces): Smooth Petal color: Immature: Upper surface: RHS 68A and RHS 67A Lower surface: RHS 65B Mature: Upper surface: RHS 68B and RHS 67A Lower surface: RHS N66D.
   Calyx.—Quantity of sepals and arrangement: Composed of 5 sepals Calyx height: 1.6 cm Calyx diameter (at the apex): 0.5 cm Sepal texture (both upper and lower surfaces): Smooth Sepal color: Upper surface: RHS 187B Lower surface: RHS 177A.
Peduncles:
   Length.—14.0 cm.
   Diameter.—0.2 cm.
   Strength.—Moderate.
   Texture.—Smooth.
   Color.—RHS 138D.
Reproductive organs:
   Stamens.—Quantity per flower: Greater than 5 Filament length: 1.0 cm Filament color: RHS 73D Anther length: 0.2 cm Anther diameter: 0.08 cm Anther color: RHS 84D Pollen amount: Moderate.
   Pistils.—Quantity per flower: 2 Pistil length: 2.0 cm Style length: 0.1 cm Style color: RHS 73D Stigma shape: Filamentous Stigma color: RHS 70B Ovary size: 0.6 cm Ovary shape: Ovate Ovary color: RHS 139D.
Fruit and seed set: No fruit and seed set observed.
Disease and pest/insect resistance: No disease and pest/insect resistance observed.

### COMPARISON WITH PARENTAL LINES AND KNOWN VARIETY

‘KLEDG13159’ is a distinct variety of Dianthus and is distinguished from its parents as described in Table 1.

TABLE 1
Comparison with Parental Lines

| | | Female Parent | Male Parent |
|---|---|---|---|
| Characteristic | ‘KLEDG13159’ | ‘DSS 207 0031’ | ‘DSS 2007 0026’ |
| Flower color | Raspberry red | Purple cerise | Pink |
| Flower type | Single | Double | Double |

‘KLEDG13159’ is a distinct variety of Dianthus and is most similar to the commercial Dianthus variety ‘KLEDG11143’ (U.S. Plant Pat. No. 23,150), also commercially known as Everlast™ ‘Burgundy Blush’. Differences between the varieties are described in Table 2.

TABLE 2
Comparison with Similar Variety

| Characteristic | ‘KLEDG13159’ | ‘KLEDG11143’ |
|---|---|---|
| Flower type | Single | Double |
| Growth | Medium | Compact |

We claim:

1. A new and distinct variety of Dianthus plant designated ‘KLEDG13159’ as shown and described herein.
